(19) United States
(12) Patent Application Publication
(10) Pub. No.: US 2023/0340129 A1
(43) Pub. Date: Oct. 26, 2023

(54) METHOD OF ENHANCING IMMUNE RESPONSE AND CANCER IMMUNOTHERAPY BY TARGETING THE CD58:CD2 AXIS

(71) Applicant: The Trustees of Columbia University in the City of New York, New York, NY (US)

(72) Inventors: Benjamin IZAR, New York, NY (US); Johannes C. MELMS, New York, NY (US); Patricia HO, New York, NY (US)

(21) Appl. No.: 18/335,826

(22) Filed: Jun. 15, 2023

Related U.S. Application Data
(63) Continuation of application No. PCT/US21/63562, filed on Dec. 15, 2021.
(60) Provisional application No. 63/125,517, filed on Dec. 15, 2020.

(51) Int. Cl. C07K 16/28; A61K 38/17

(52) U.S. Cl. CPC C07K 16/2827; A61K 38/1774

(57) ABSTRACT

The method of enhancing anti-tumor immunity in a patient by targeting the CD58:CD2 axis uses an administered treatment to target and disrupt CMTM6 regulation of PD-L1 protein, thus enhancing the immune response and cancer immunotherapy in the patient. Targeting and disruption of CMTM6 regulation of the PD-L1 protein may be combined with additional prompting of a PD-1 blockage or adoptive cell transfer (ACT) in the patient. Targeting and disrupting CMTM6 regulation of PD-L1 protein may be initiated by administering an effective amount of antibodies to the patient, which are specific to disrupting CMTM6/PD-L1 protein interaction. Alternatively, to enhance the anti-tumor immunity in the patient, CD2 mediated signaling may be increased in order to stimulate an immune response. As another alternative to using antibodies or a CD58 mimetic, a pharmacological target to boost CD2/CD58 signaling in the patient may be identified and administered to enhance anti-tumor immunity.

### CROSS-REFERENCE TO RELATED APPLICATIONS

This application is a continuation of International Patent Application No. PCT/US2021/063562, filed on Dec. 15, 2021, titled “Method of Enhancing Immune Response And Cancer Immunotherapy By Targeting the CD58:CD2 Axis,” which claims the benefit of U.S. Provisional Patent Application No. 63/125,517, filed on Dec. 15, 2020, the entirety of the disclosures of which are hereby incorporated by this reference.

### GOVERNMENT LICENSE RIGHTS

This invention was made with government support under grant CA222663 awarded by the National Institutes of Health. The government has certain rights in the invention.

### TECHNICAL FIELD

The disclosure of the present patent application relates to activating the CD2 receptor on CD28-CD8+T cells to facilitate and stimulate immune response and cancer immunotherapy, thereby enhancing anti-tumor immunity in a patient.

### BACKGROUND ART

Cancer immunotherapies have revolutionized the clinical care of cancer patients. The most effective immunotherapy is the use of anti-PD-1 antibodies. The effectiveness of these therapies depends on expression of CD28 on CD8⁺ T cells (CD28⁺ CD8⁺ T cells). Yet, in most cancer patients, tumors have a large fraction of CD28-CD8⁺ T cells, rendering them insensitive to such revolutionary therapies.

Prior studies identified CD2 on CD8⁺ T cells as the most potent activator in the context of lack of CD28 expression. The ligand for CD2 is CD58. CD58 is typically expressed on antigen presenting cells, such as macrophages, yet the role of CD58 on cancer cells has, thus far, remained unknown. To date, no therapies have been developed to specifically activate CD28⁻CD8⁺ T cells. Alternative avenues for activating the ability of CD8⁺ T cells to promote anti-tumor immunity are desperately needed and would expand potential benefits to hundreds of thousands of cancer patients. Thus, a method of enhancing anti-tumor immunity in a patient by targeting the CD58:CD2 axis solving the aforementioned problems is desired.

### DISCLOSURE

The method of enhancing anti-tumor immunity in a patient by targeting the CD58:CD2 axis uses an administered treatment to target and disrupt CMTM6 regulation of PD-L1 protein in the patient, thus enhancing the patient's immune response and cancer immunotherapy. The targeting and disruption of CMTM6 regulation of the PD-L1 protein may be combined with additional prompting of a PD-1 blockage or adoptive cell transfer (ACT) in the patient. The targeting and disrupting CMTM6 regulation of PD-L1 protein in the patient may be initiated by administering an effective amount of antibodies to the patient, where the antibodies are specific to disrupting CMTM6/PD-L1 protein interaction.

Alternatively, in order to enhance the anti-tumor immunity in the patient, CD2 mediated signaling in the patient may be increased in order to stimulate an immune response. The increase of the CD2 mediated signaling in the patient may be initiated by administering an effective amount of antibodies to the patient, where the antibodies are specific to activating CD2 to increase the CD2 mediated signal. As a further alternative, CD58 mimetics may be administered to the patient in order to increase the CD2 mediated signal. Direct stimulation of CD2 stimulates immune responses. Thus, by using antibodies, CD58 mimetics, or any other suitable means for activating the CD2 receptor on CD28-CD8⁺ T cells, potent cancer immunotherapy options and alternatives are provided.

As a further alternative, rather than using antibodies or a CD58 mimetic, a pharmacological target to boost CD2/CD58 signaling in the patient may be identified. Then, an effective amount of at least one pharmacological agent may be administered to the patient, where the at least one pharmacological agent is specific to the identified pharmacological target to boost CD2/CD58 signaling in the patient and stimulate an immune response.

These and other features of the present subject matter will become readily apparent upon further review of the following specification.

### BRIEF DESCRIPTION OF THE DRAWINGS

FIG. 1 diagrammatically illustrates the role of CD58 in the tumor immune synapse of a sensitive cancer cell.

FIG. 2 diagrammatically illustrates the primary role of CD28 as a co-stimulatory signal in APC:T cell interactions.

FIG. 3A is a graph comparing measured interferon-γ(IFN-γ) release from CD8⁺/CD28⁻ T cells after stimulation with mouse thymoma cells stably expressing anti-human CD3 antibody fragment and costimulatory ligands (CD80, CD58, 41BBL, MICA, ICOSL, CD166, CD54, CD70) or no costimulatory ligand (control). Units in ng/ml.

FIG. 3B is a graph comparing measured interleukin-2 (IL-2 release from CD8⁺/CD28⁻ T cells after stimulation with mouse thymoma cells stably expressing anti-human CD3 antibody fragment and costimulatory ligands (CD80, CD58, 41BBL, MICA, ICOSL, CD166, CD54, CD70) or no costimulatory ligand (control). Units in ng/ml.

FIG. 4A shows the results of CD28 expression on CD8 tumor infiltrating lymphocytes (TILs) from four representative patients.

FIG. 4B shows the percent of CD28⁺ cells among CD8 tumor infiltrating lymphocytes (TILs) from 17 patients.

FIG. 4C compares expression of CD2 and CD28 in tumor infiltrating lymphocytes (TILs) in human tumors profiled by single cell RNA sequencing.

FIG. 5A shows the distribution of fluorescent intensity of CD58 (APC-CD58), B2M (APC-B2M), PD-L1 (APC-CD274) or staining with isotype control, without or with IFN-γ stimulation, in patient-derived melanoma cells, validating the KO of B2M (left plot), PD-L1 (middle plot) and CD58 (right plot).

FIG. 5B shows graphs comparing the ratios of viable cancer cells in patient-derived TIL:melanoma co-culture models of control, CD58 knockout (KO), beta-2-microglobulin (B2M) KO and CD274 KO cells.

FIG. 6A diagrammatically illustrates the experimental design measuring CD58 knockout (KO) cells (red fluorescent protein+, RFP) outcompeting parental cells (blue fluorescent protein+, BFP) during T cell mediated selection pressure.

FIG. 6B is a graph showing the relative enrichment effect of KO cells (RFP) over control target cells (BFP+) for CD58 KO cells, B2M KO cells, CD274K0 cells, and unmodified cells (control) after coculture with cytotoxic TILs.

FIG. 7A is a graph illustrating the comparable growth of control and KO cells, showing the ratio of viable cells relative to timepoint 0 for control and B2M KO, CD58 KO or CD274 KO melanoma cells.

FIG. 7B is a graph illustrating the comparable induction of apoptosis in response to Staurosporin and resistance to DTIC in control and KO melanoma cells, showing the percent of cells inducing Caspase 3/7 in control and B2M KO, CD58 KO or CD274 KO melanoma cells in different treatment conditions.

FIG. 8A is a graph showing the surface expression of MHC class-I, both at baseline and after stimulation with different levels of IFN-γ for 72 hours, in parental and CD58 KO cells.

FIG. 8B is a graph showing the surface expression of MHC class-II, both at baseline and after stimulation with different levels of IFN-γ for 72 hours, in parental and CD58 KO cells.

FIG. 9A is a graph comparing CD58 KO cells against a control, CD58 transcript 1 rescue (glycosylphosphatidylinositol (GPI)-anchored form), and CD58 transcript 2 rescue (transmembrane (TM) form), and showing that re-expression of either CD58 isoform rescues sensitivity to TIL mediated killing.

FIG. 9B shows a distribution of expression levels of CD58 RNA in melanoma cells from tumors in patients who were either treatment naïve (TN) or were resected after failure of immunotherapy in the scRNA-seq data from the ICR-signature discovery cohort.

FIG. 10A diagrammatically illustrates the experimental design of a partially humanized mouse model comparing growth and TIL infiltration in tumor grafts of CD58 WT and CD58 knock-out tumors.

FIG. 10B is a graph comparing tumor-related results from both the CD58 WT and CD58 KO models of FIG. 10A, showing that CD58 KO confers exclusion of T cells from the tumor microenvironment (TME) and resistance to adoptive cell transfer (ACT) in the partially humanized mouse model.

FIG. 11A shows the regulatory effect in Perturb-CITE-Seq on key RNA and protein (CITE) features when perturbing different genes in the JAK-STAT pathway, CD58 or CD274.

FIG. 11B is a graph showing measured geometric mean fluorescence intensity (gMFI) values for PD-L1 expression, comparing CD58 KO against a control, specifically showing the results at baseline and after stimulation with different levels of IFN-γ for 72 hours.

FIG. 11C is a graph showing measured gMFI values for PD-L1 expression following IFN-γ induction, comparing CD58 KO against a control, CD58-1 rescue, and CD58-2 rescue.

FIG. 12 at top, shows immunoblotting for PD-L1 in immunoprecipitation of CMTM6 and associated proteins, demonstrating that CMTM6 binds to PD-Ll. In middle is immunoblotting for CMTM6 in parental, CD58 KO, and CMTM6 KO melanoma cells. At bottom is shown immunoblotting of CD58 in immunoprecipitation of CMTM6 and associated proteins in both WT and CMTM6 KO melanoma cells.

FIG. 13A is a graph showing measured gMFI values for PD-L1 expression following interferon-γ (IFN-γ) induction, comparing CMTM6 KO against a control after 72 hours.

FIG. 13B is a graph showing measured GMF values for CD58 expression following interferon-γ (IFN-γ) induction, comparing CMTM6 KO against a control after 72 hours.

FIG. 14 diagrammatically illustrates a proposed scheme for how PD-L1 and CD58 compete for CMTM6 (illustrated as oval circles in membrane associating with PD-L1 and CD58 receptors).

FIG. 15A diagrammatically illustrates how PD-L1 competes with the RNA exosome to regulate the DNA damage response and can be targeted to sensitize to radiation or chemotherapy.

FIG. 15B diagrammatically illustrates the H1A binding epitope on PD-L1.

FIG. 15C shows western blots illustrating control and CMTM6 knockout cells treated with either IgG or H1A (20 μg/mL) antibody, comparing PD-L1 protein levels.

Similar reference characters denote corresponding features consistently throughout the attached drawings.

### DESCRIPTION OF EMBODIMENTS

The method of enhancing anti-tumor immunity in a patient by targeting the CD58:CD2 axis uses an administered treatment to target and disrupt CMTM6 regulation of PD-L1 protein in the patient, thus enhancing anti-tumor immunity in the patient. The targeting and disruption of CMTM6 regulation of the PD-L1 protein may be combined with additional prompting of a PD-1 blockage or adoptive cell transfer (ACT) in the patient. The targeting and disruption of CMTM6 regulation of PD-L1 protein in the patient may be initiated by administering an effective amount of antibodies to the patient, where the antibodies are specific to disrupting CMTM6/PD-L1 protein interaction.

Alternatively, in order to enhance the anti-tumor immunity in the patient, CD2 mediated signaling in the patient may be increased in order to stimulate an immune response. The increase of the CD2 mediated signaling in the patient may be initiated by administering an effective amount of antibodies to the patient, where the antibodies are specific to activating CD2 to increase the CD2 mediated signal. As a further alternative, CD58 mimetics may be administered to the patient in order to increase the CD2 mediated signal. Direct stimulation of CD2 stimulates immune responses. Thus, by using antibodies, CD58 mimetics, or any other suitable means for activating the CD2 receptor on CD28⁻CD8⁺ T cells, potent cancer immunotherapy options and alternatives are provided.

As a further alternative, rather than using antibodies or a CD58 mimetic, a pharmacological target to boost CD2/CD58 signaling in the patient may be identified. Then, an effective amount of at least one pharmacological agent may be administered to the patient, where the at least one pharmacological agent is specific to the identified pharmacological target to boost CD2/CD58 signaling in the patient and stimulate an immune response.

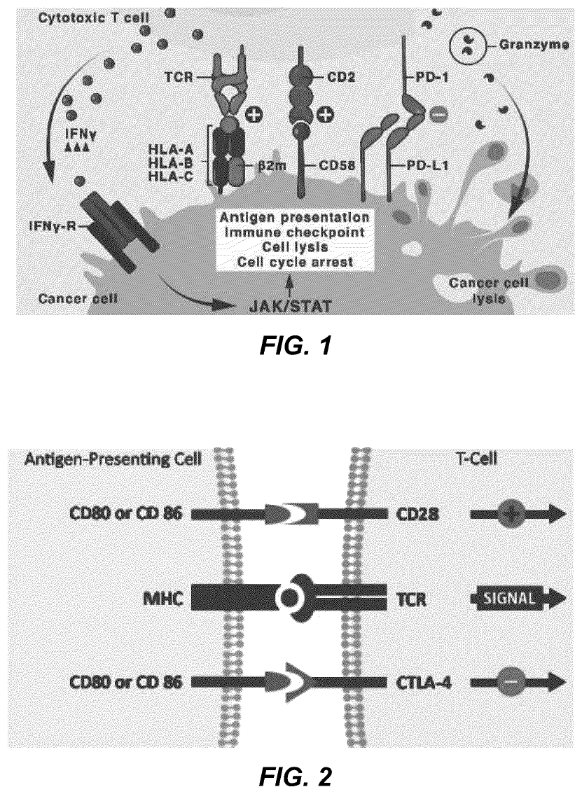

CD58 is expressed on cancer cells and is both sufficient and necessary for promoting anti-tumor immunity in CD28⁻CD8⁺ T cells. Genetic ablation of CD58 on cancer cells renders these cells completely resistant to anti-tumor immunity. Re-expression of CD58 in CD58 knockout cancer cells in two different biochemical anchorages re-sensitizes cancer cells to anti-tumor immunity. FIG. 1 illustrates the role of CD58 in the tumor immune synapse of a sensitive cancer cell. It is noted that CD58 is recurrently and concordantly regulated across analytes. CD58/CD2 is the primary costimulatory pathway in human CD28⁻CD8⁺ T cells. As can be seen in the diagram of interactions between T cells and cancer cells, at baseline, T cell receptor (TCR) stimulation via peptide-loaded MHC Class I and through CD58:CD2 co-stimulation results in production of cytokines (such as IFN-γ, granzymes), which lead to activation of the IFN-γ-JAK/STAT-pathway that determines the cell fate and expression of surface proteins.

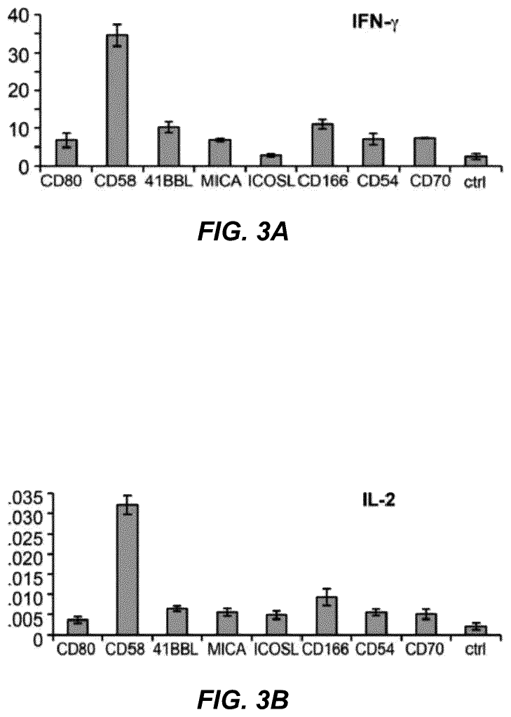

As illustrated in FIGS. 2, 3A and 3B, in the absence of CD28 co-stimulation, the CD58:CD2 axis is the most potent co-stimulatory signal in APC:T cell interactions. FIG. 2 illustrates the role of CD28 co-stimulation. FIG. 3A compares measured interferon-γ (IFN-γ) results for CD28⁻CD8⁺ T cells after costimulation via CD80, CD58, 41BBL, MICA, ICOSL, CD166, CD54, and CD70 using artificial antigen presenting cells lacking CD28 co-stimulation. Similarly, FIG. 3B compares measured interleukin-2 (IL-2) results for costimulation via CD80, CD58, 41BBL, MICA, ICOSL, CD166, CD54, and CD70 using artificial antigen presenting cells lacking CD28 co-stimulation. It can be seen that CD58 produces the greatest immune response.

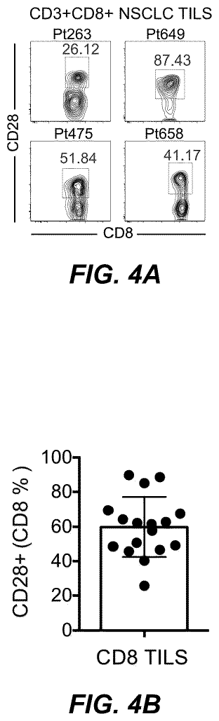

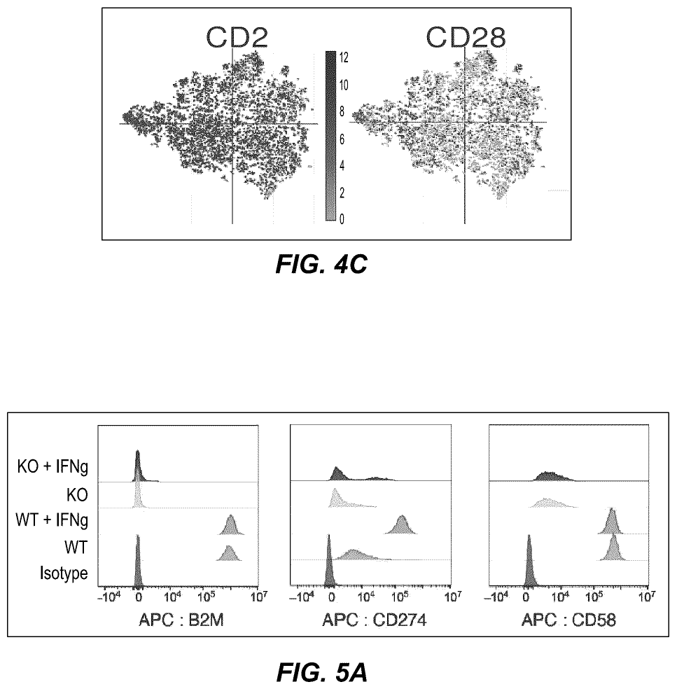

Further, FIG. 4A shows the results of CD28 expression on CD8 tumor infiltrating lymphocytes (TILs) from four representative patients, and FIG. 4B shows similar results but for 17 patients (where the error bars indicate the standard error of the mean (SEM)). As shown, the expression of CD28 on the CD⁸⁺ TILs is highly variable in non-small cell lung cancer (NSCLC) and melanoma, while, as shown in FIG. 4C, CD2 expression is largely preserved.

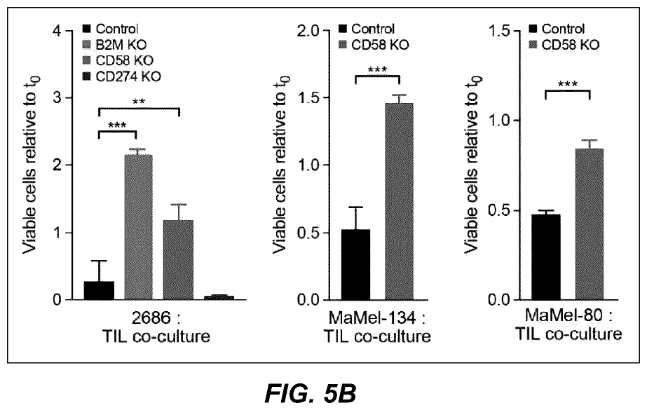

Additionally, FIGS. 5A and 5B compare loss of CD58 against loss of B2M and CD274 for three different patient models, showing that loss of CD58 confers immune evasion across each different patient model. FIG. 5A shows the distribution of fluorescent intensity of CD58 (APC-CD58), B2M (APC-B2M), PD-L1 (APC-CD274) or staining with isotype control, without or with IFN-γ stimulation, in WT and respective KO versions of patient-derived melanoma cells (#2686). FIG. 5B shows graphs comparing the ratios of viable cancer cells in cancer-immune co-culture models of control, CD58 knockout (KO), beta-2-microglobulin (B2M) KO and CD274 KO target cells.

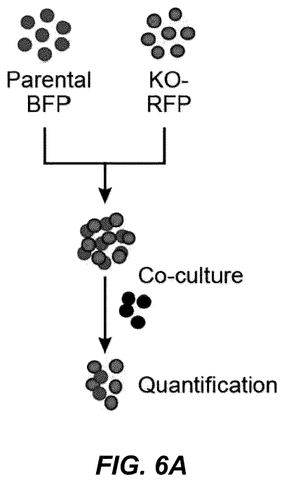

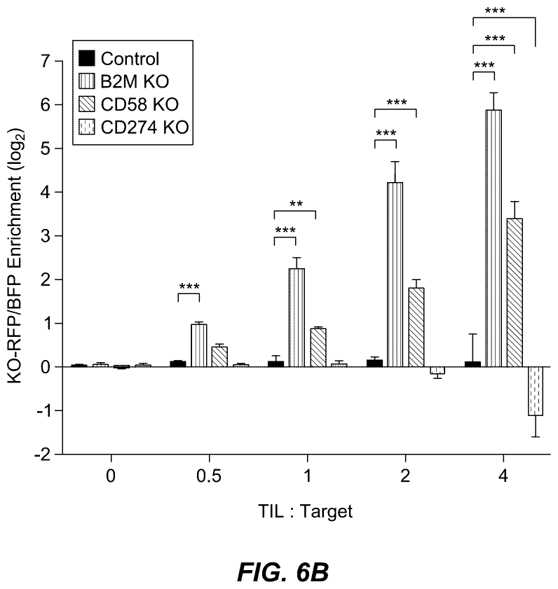

FIG. 6A diagrammatically illustrates the experimental design measuring CD58 KO cells (labeled with RFP) outcompeting parental cells (labeled with BFP) during T cell mediated selection pressure, and FIG. 6B shows this effect for CD58 KO compared against B2M KO, CD274 KO and a control. In FIG. 6A, BFP-labeled parental cells and RFP-labeled KO cells are co-cultured with TILs and the RFP/BFP ratio is calculated as an estimate of relative fitness. FIG. 6B shows a competition assay of parental cells and matched B2M KO, CD58 KO or CD274 KO after 48 hours of co-culture.

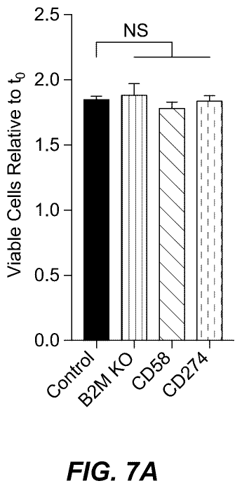

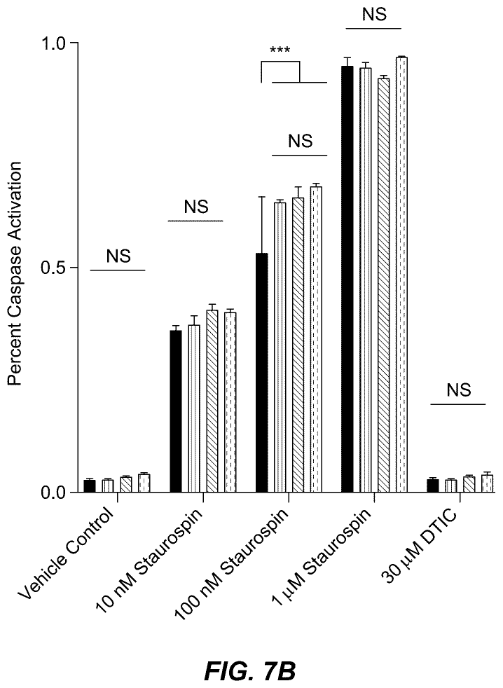

Further, FIG. 7A shows proliferation results comparing CD58 loss against B2M loss, CD274 loss and a control, and FIG. 7B shows associated results for apoptotic potential to test non-specific resistance. FIGS. 7A and 7B illustrate that the competitive advantage of CD58 KO cells is not due to differences in proliferation, apoptotic potential or sensitivity/resistance to non-specific treatments. FIG. 7A illustrates the comparable growth of control and KO cells, showing the ratio of viable cells relative to timepoint 0 for control and B2M KO, CD58 KO or CD274 KO melanoma cells from patient 2686. FIG. 7B illustrates the comparable induction of apoptosis in response to Staurosporin and resistance to DTIC in control and KO melanoma cells, showing the percent of cells inducing Caspase 3/7 in control, B2M KO, CD58 KO or CD274 KO patient-derived melanoma cells in different treatment conditions.

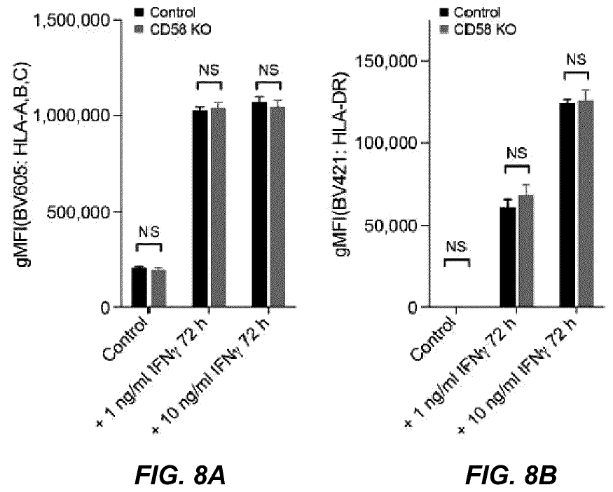

FIGS. 8A and 8B show a comparison between CD58 KO cells and a control, showing that CD58 KO cells do not impair major histocompatibility complex (MHC) I/II expression at baseline and induction. It is clear that CD58 loss does not impair MHC expression. FIG. 8A shows the surface expression of MHC class-I, and FIG. 8B shows the surface expression of MHC class-II, both at baseline and after stimulation with different levels of IFN-γ for 72 hours, in parental and CD58 KO cells.

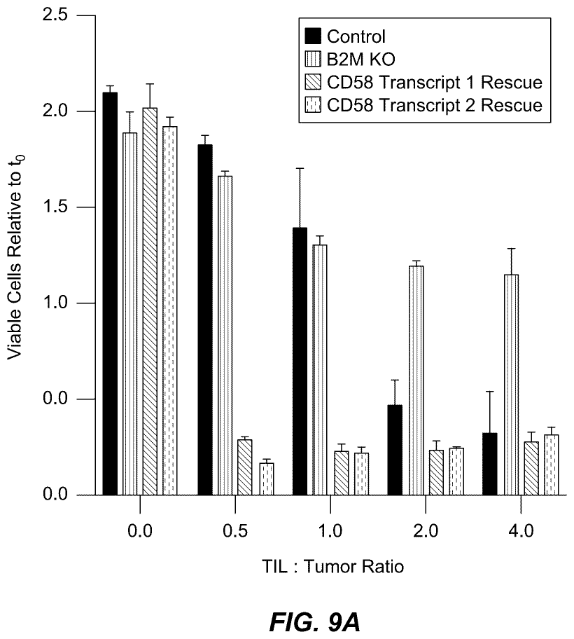

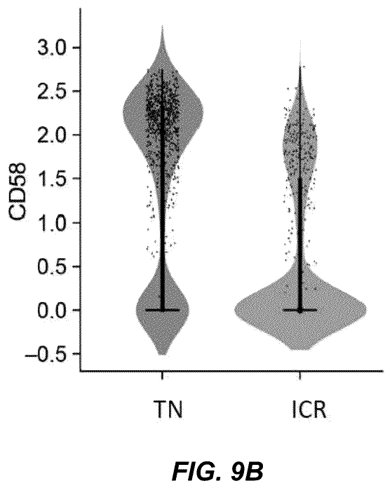

FIG. 9A compares CD58 KO cells against a control, CD58 transcript 1 rescue, and CD58 transcript 2 rescue, showing that re-expression of CD58 (both the glycosylphosphatidylinositol (GPI)-anchored form and a transmembrane (TM) form) rescue the sensitivity phenotype. FIG. 9B shows a distribution of expression levels of CD58 RNA in melanoma cells from tumors in patients who were either treatment naïve (TN) or were resected after failure of immunotherapy in the scRNA-seq data from the ICR-signature discovery cohort. FIGS. 9A and 9B illustrate that CD58 downregulation is associated with immune checkpoint inhibitor resistance (ICR) in melanoma.

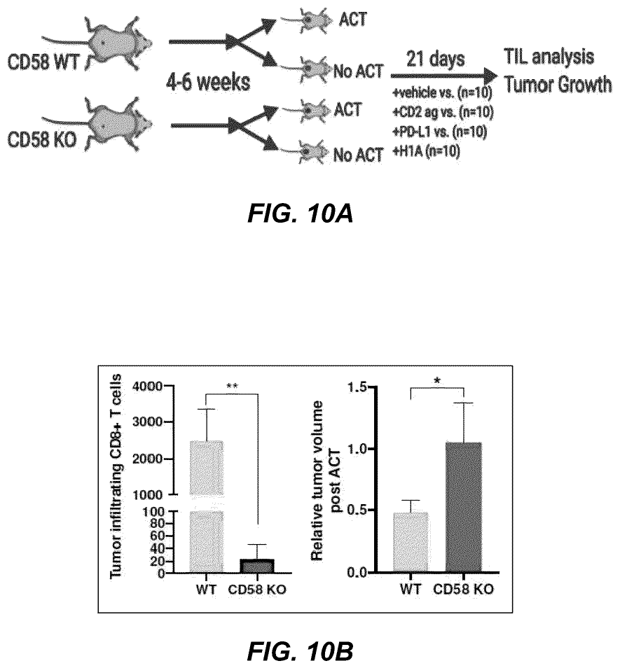

Because a CD58 homolog does not exist in mouse models that are typically used for studying immunotherapies, we established humanized mouse models and confirmed the role of CD58 in vivo. FIG. 10A diagrammatically illustrates the experimental design of a partially humanized mouse model comparing growth and TIL infiltration in tumor grafts of CD58 WT and CD58 knock-out tumors. FIG. 10B compares tumor-related results from both the CD58 WT and CD58 KO models, showing that CD58 KO confers exclusion of T cells from the tumor microenvironment (TME) and resistance to adoptive cell transfer (ACT) in the partially humanized mouse model. From the above, it can be seen that CD58 loss/downregulation is associated with immune evasion through impaired T cell mediated tumor lysis, T cell exclusion, and resistance to ACT.

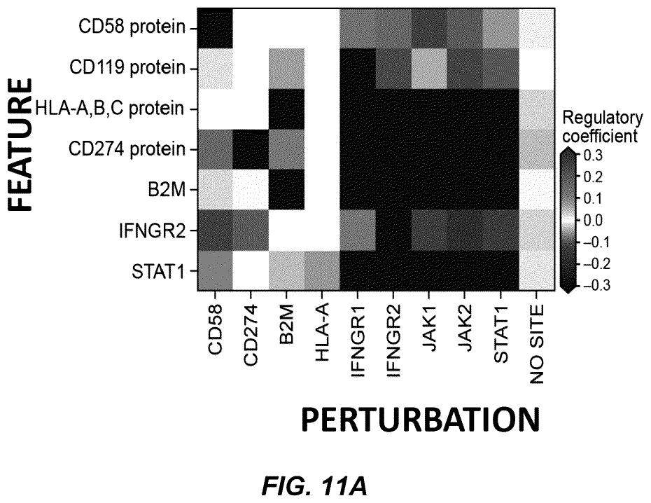

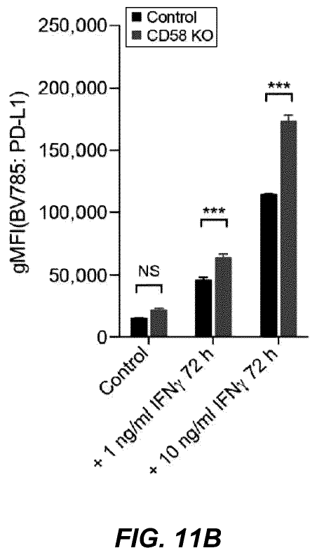

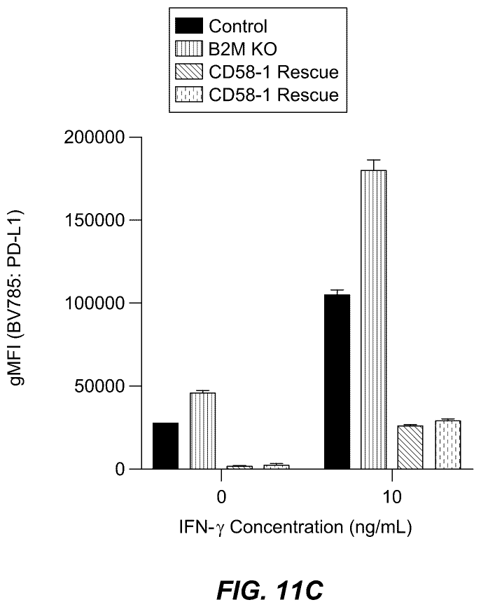

FIG. 11A shows that CD58 perturbation in co-culture does not affect B2M and HLA expression at the RNA and protein level but induces CD274. Specifically, FIG. 11A shows the regulatory effect in Perturb-CITE-Seq on key RNA and protein (CITE) features when perturbing different genes in the JAK-STAT pathway, CD58 or CD274. FIG. 11B shows measured geometric mean fluorescence intensity (gMFI) values for PD-L1 expression following interferon-γ (IFN-γ) induction, comparing CD58 KO against a control. Specifically, FIG. 11B shows the results for surface expression of CD274 at baseline and after stimulation with different levels of IFN-γ for 72 hours, in parental and CD58 KO cells. FIG. 11C shows measured gMFI values for PD-L1 expression following IFN-γ induction, comparing CD58 KO against a control, CD58-1 rescue, and CD58-2 rescue. It can be seen that the loss of CD58 results in increased PD-L1 expression following IFN-γ induction. From the above, it can also be seen that CD58 loss/downregulation is associated with immune evasion through upregulation of PD-L1, and is also associated with ICR in patients. Additionally, it can be seen that CD58 expression is not impaired in defects of the IFN-γ-JAK/STAT pathway.

We have demonstrated that CD58 loss confers resistance to immunity also by activating inhibitory pathways, such as the PD-L1/PD-1 pathway. We propose that CD58 loss enhances PD-L1 signaling by releasing CMTM6, thereby enhancing PD-L1 protein stability. Targeted disruption of compensatory PD-L1 expression, such as by using antibodies that destabilize the CMTM6/PD-L1 protein interaction, releasing CMTM6 to stabilize CD58, canthereby enhance anti-tumor immunity.

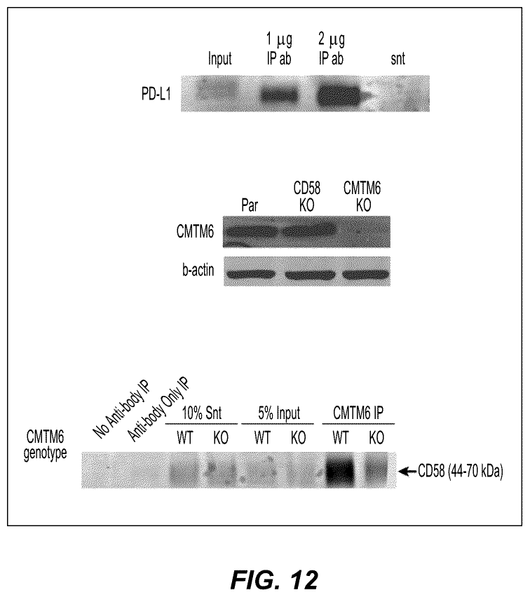

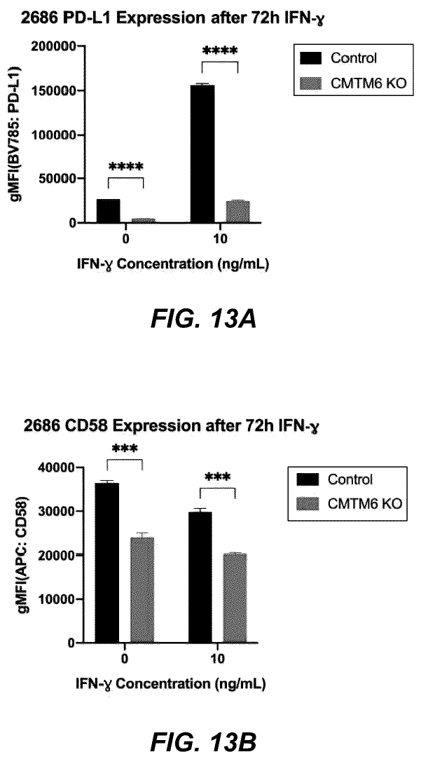

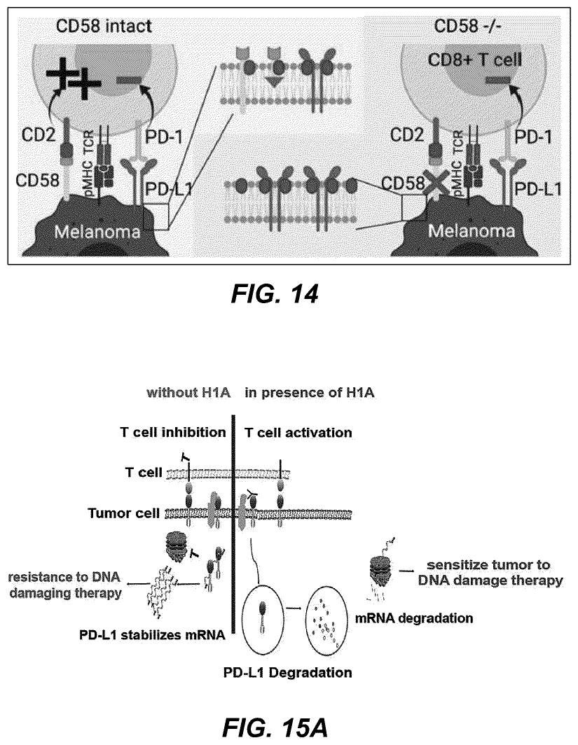

CMTM6 is a ubiquitously expressed, protein that binds PD-L1 and maintains its cell surface expression. CMTM6 is not required for PD-L1 maturation but co-localizes with PD-L1 at the plasma membrane and in recycling endosomes where it prevents PD-L1 from being targeted for lysosome-mediated degradation. As shown in the blots of FIG. 12, CMTM6 is found to interact with both CD58 and PD-L1. FIG. 13A and 13B show measured gMFI values for PD-L1 expression following interferon-γ (IFN-γ) induction, comparing CMTM6 KO against a control after 72 hours for patient 2686. FIGS. 13A and 13B show that the loss of CMTM6 results in reduced expression of PD-L1 and CD58. FIG. 14 diagrammatically illustrates a proposed scheme for how PD-L1 and CD58 compete for CMTM6.

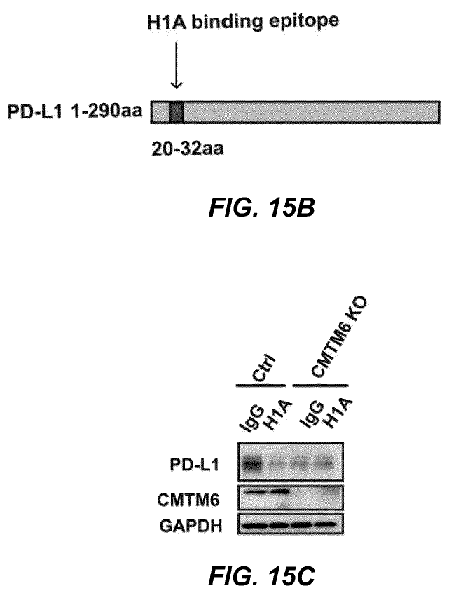

FIG. 15A diagrammatically illustrates how PD-L1 competes with the RNA exosome to regulate the DNA damage response and can be targeted to sensitize to radiation or chemotherapy. The anti-PD-L1 antibody H1A has been found to destabilize PD-L1 and sensitize cancer to radiotherapy. FIG. 15B diagrammatically illustrates the H1A binding epitope on PD-L1. FIG. 15C shows control and CMTM6 knockout cells treated with either IgG or H1A (20 μg/mL) antibody, with the PD-L1 level being determined by western blot.

The above shows that CMTM6 interacts with CD58, and that CMTM6 KO results in reduced surface expression of PD-L1 and CD58. It has been further shown that PD-L1 and CD58 may compete for CMTM6. Thus, pharmacological strategies to release CMTM6 while degrading PD-L1 may be effective in the context of PD-L1 associated immune evasion.

It is to be understood that the method of enhancing anti-tumor immunity in a patient by targeting the CD58:CD2 axis is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

What is claimed is:

1. A method of enhancing anti-tumor immunity in a patient by targeting the CD58:CD2 axis, comprising targeting and disrupting CMTM6 regulation of PD-L1 protein in the patient to enhance immune response and cancer immunotherapy in the patient.

2. The method of enhancing anti-tumor immunity in a patient by targeting the CD58:CD2 axis as recited in claim 1, further comprising the step of prompting a PD-1 blockade or adoptive cell transfer (ACT) in the patient.

3. The method of enhancing anti-tumor immunity in a patient by targeting the CD58:CD2 axis as recited in claim 1, wherein the step of targeting and disrupting CMTM6 regulation of PD-L1 protein in the patient comprises administering an effective amount of antibodies to the patient, wherein the antibodies are specific to disrupting CMTM6/PD-L1 protein interaction.

4. A method of enhancing anti-tumor immunity in a patient by targeting the CD58:CD2 axis, comprising increasing CD2 mediated signaling in the patient to stimulate an immune response in the patient.

5. The method of enhancing anti-tumor immunity in a patient by targeting the CD58:CD2 axis as recited in claim 4, wherein the step of increasing CD2 mediated signaling in the patient comprises administering an effective amount of antibodies to the patient, wherein the antibodies are specific to activating CD2 to increase the CD2 mediated signal.

6. The method of enhancing anti-tumor immunity in a patient by targeting the CD58:CD2 axis as recited in claim 4, wherein the step of increasing CD2 mediated signaling in the patient comprises administering an effective amount of CD58 mimetics to the patient to increase the CD2 mediated signal.

7. A method of enhancing anti-tumor immunity in a patient by targeting the CD58:CD2 axis, comprising the steps of:
identifying a pharmacological target to boost CD2/CD58 signaling in the patient; and
administering an effective amount of at least one pharmacological agent to the patient, wherein the at least one pharmacological agent is specific to the identified pharmacological target to boost CD2/CD58 signaling in the patient and stimulate an immune response in the patient.
